(19) United States
(12) Patent Application Publication
(10) Pub. No.: US 2025/0228890 A1
(43) Pub. Date: Jul. 17, 2025

(54) ANTI-CANCER FORMULATION COMPRISING SODIUM PENTABORATE, CURCUMIN AND PIPERINE FOR USE IN THE TREATMENT OF HEPATOCELLULAR CARCINOMA

(71) Applicant: Yeditepe Universitesi, Istanbul (TR)

(72) Inventors: Fikrettin SAHIN, Atasehir/Istanbul (TR); Zehra OMEROGLU ULU, Atasehir/Istanbul (TR); Zeynep Busra BOLAT, Küçükçekmece/Istanbul (TR); Nurdan Sena DEGIRMENCI, Istanbul (TR)

(73) Assignee: Yeditepe Universitesi, Istanbul (TR)

(21) Appl. No.: 18/872,899

(22) PCT Filed: Mar. 23, 2023
(86) PCT No.: PCT/TR2023/050276

§ 371 Date: Dec. 9, 2024

(30) Foreign Application Priority Data
Mar. 31, 2022 (TR) 2022/005058

(51) Int. Cl. A61K 33/22; A61K 31/12; A61K 31/4525; A61K 36/67; A61K 36/9066; A61P 35/00

(52) U.S. Cl. CPC A61K 33/22; A61K 31/12; A61K 31/4525; A61K 36/67; A61K 36/9066; A61P 35/00

(57) ABSTRACT

The present invention relates to a formulation together comprising sodium pentaborate, curcumin, and piperine as active substances and to use thereof in the treatment of hepatocellular carcinoma.

### FIELD OF THE INVENTION

The present invention relates to a formulation comprising sodium pentaborate, curcumin, and piperine as active substances and to use thereof in the treatment of hepatocellular carcinoma.

### BACKGROUND OF THE INVENTION

Hepatocellular carcinoma is the most common malignant primary liver tumor, which originates from hepatocytes and occurs in the setting of chronic liver disease usually caused by viral hepatitis. Globally, it is the fifth most common cancer and the third most common cause of death from cancer. The incidence and prevalence of hepatocellular carcinoma has been observed to increase rapidly in our country with the increase in chronic hepatitis [1-4]. The large majority of hepatocellular carcinoma cases occur in the setting of chronic liver disease, with cirrhosis being the primary risk factor for hepatocellular carcinoma independent of liver disease etiology. It is estimated that one-third of cirrhotic patients will develop liver cancer during their lifetime, with a 1-8% annual incidence reported in long-term follow-up studies. The incidence of hepatocellular carcinoma appears to be lower in alcoholic and non-alcoholic steatohepatitis-related cirrhosis than active viral hepatitis, but the incidence appears to be greater than 1.5% in cirrhosis etiologies [5, 6].

There is currently no effective treatment method for hepatocellular carcinoma. Liver transplantation or surgical resection is implemented in patients with early-stage tumor, while chemotherapy and/or radiation are the current treatment modalities for patients with advanced-stage tumor. Although liver transplantation and resection are the only curative treatments, these two therapies can only be implemented in a small percentage of patients. One of the reasons for this is that hepatocellular carcinoma is often diagnosed at unresectable stages. Recurrence after resection is observed in more than 80% of patients [7].

Boron derivatives are encountered in many areas today. Investigation of the use of boron derivatives as anti-cancer agents in cancer treatment has been a promising new application area for cancer therapies [8, 9]. Boron derivatives have been demonstrated to be effective in hepatocellular carcinoma. For example, studies have been conducted, in which cancer cells in hepatocellular carcinoma can be selectively captured and destroyed by boron neutron capture therapy [10, 11]. In addition, HepG2 cells, a hepatocellular carcinoma cell line, were treated with boric acid, a boron derivative, and a microarray experiment was performed to examine the affected pathways. It was revealed that boric acid treatment induced anti-cancer mechanisms [12]. Bortezomib, an FDA-approved proteasome inhibitor which is used in cancer treatment, is also a pyrazine and boronic acid derivative. It has also shown clinical success in the treatment of multiple myeloma and mantle cell lymphoma, as well as colon, lung, breast and prostate cancers and hepatocellular carcinoma. Despite all these clinical successes, there are limitations to this drug, as some patients treated with bortezomib have had cancer recurrence or have not responded to treatment at all. Resistance to bortezomib has been observed in some solid tumors [13-17].

While sodium pentaborate pentahydrate (Na₂B₁₀O₁₆·5H₂O, 18% B; NaB: B₅H₁₀NaO₁₃) is known to promote wound healing, a study has shown that it has anti-cancer activity in lung cancer [18-19]. Furthermore, in a study which was conducted by using hepatocellular carcinoma Hep3B cell line, it was shown that sodium pentaborate pentahydrate decreased the proliferation of Hep3B cells and led to metabolic changes through the activation of SIRT3 gene [20].

Curcumin is a hydrophobic polyphenol naturally extracted from the rhizome of the Curcuma longa (turmeric) plant. Curcumin has versatile metabolic effects, including anti-oxidant, anti-inflammatory, anti-viral, anti-angiogenic, anti-microbial, and anti-cancer activities. Several clinical trials classify curcumin as a potential chemopreventive and chemotherapeutic agent. Although curcumin is a non-toxic substance, it shows poor bioavailability. This problem has been observed to be eliminated when curcumin treatment is performed with some secondary agents [21-23]

Piperine is another polyphenol which is isolated from black long peppers and is distinguished by its unique properties. It not only improves the existing anti-cancer activity of curcumin, but also increases the bioavailability of curcumin. It has been shown that when curcumin and piperine are administered together, it increases the anti-cancer activity of curcumin in hepatocellular carcinoma, colorectal, leukemia, and breast cancers [24-27].

There is a need for new drug derivatives due to the development of drug resistance mechanisms.

Currently, drugs approved by the FDA for the treatment of hepatocellular carcinoma include Sorafenib, Bevacizumab, Cabozantinib, and Regorafenib (ClinicalTrials.gov; cancer.gov). These drugs, which have become part of routine treatment, have problems in terms of resistance in the course of the disease and do not have a significant positive effect on survival.

Patent application no. EP1790349A1 known in the state of art discloses the treatment of viral or bacterial infections with pentahydrated sodium pentaborate {NaB5O2·5H2O}. However, there is no mention of the effects of the component in the treatment of diseases and/or its combined use.

By combining drugs that activate different mechanisms, cancer cells can be prevented from becoming resistant. Moreover, since the dose delivered in drug combinations is lower than the optimal dose delivered alone, the cytotoxic effects of each drug are eliminated.

### SUMMARY OF THE INVENTION

The present invention relates to a ternary combination comprising a boron derivative and at least two polyphenol active substances used for the treatment of hepatocellular carcinoma. Here, the boron derivative is preferably sodium pentaborate and the polyphenol active substances are curcumin and piperine.

The present invention relates to a pharmaceutical formulation comprising sodium pentaborate pentahydrate, curcumin, and piperine or pharmaceutically acceptable salts or derivatives thereof, which provides additional advantages to the field of the art.

The main objective of the invention relates to a pharmaceutical formulation comprising sodium pentaborate pentahydrate, curcumin, and piperine or pharmaceutically acceptable salts or derivatives thereof, which is used in the treatment of hepatocellular carcinoma.

The invention relates to creating a synergistic effect by combining drugs that activate different mechanisms (cell pathways). Thus, cancer cells are prevented from becoming resistant. Moreover, since the dose delivered in drug combinations is lower than the optimal dose delivered alone, the cytotoxic effects of each drug are eliminated.

### BRIEF DESCRIPTION OF THE FIGURES

“An Anti-Cancer Formulation Comprising Sodium Pentaborate, Curcumin and Piperine for Use in the Treatment of Hepatocellular Carcinoma” which was developed for achieving the objective of the present invention, is illustrated in the accompanying figures, in which:

FIG. 1 shows the results of MTS analysis of Hep3B cells, which were treated with sodium pentaborate pentahydrate (A) curcumin (B), piperine (C), and the combinations thereof (D), based on cell viability percentages.

FIG. 2 shows the results of MTS analysis of HepG2 cells, which were treated with sodium pentaborate pentahydrate (A) curcumin (B), piperine (C), and the combinations thereof (D), based on cell viability percentages.

FIG. 3 shows the results of 48-hour MTS analysis of HUVEC cells treated with ternary combinations based on cell viability percentages.

FIG. 4 shows the percentages of cell death leading to apoptosis in HepG2 and Hep3B cells, which were treated with sodium pentaborate pentahydrate (SPP), curcumin (Cur), and piperine (Pip), based on apoptotic cell death.

FIG. 5 shows the number of genes with increased and decreased expression levels in HepG2 and Hep3B cells, which were treated with sodium pentaborate pentahydrate, curcumin, and piperine, based on RNA sequencing. In the study conducted with RNA sequencing technology, combined drug administration caused a significant dysregulation in gene expression levels in both cell lines.

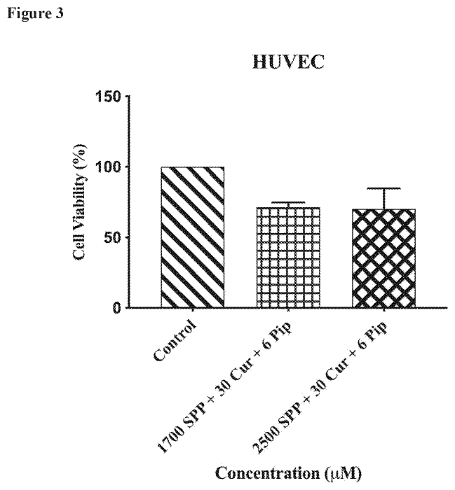

FIG. 6 shows the result of the analysis in GO and KEGG databases of genes whose expression increased with drug combination in HepG2 cells.

FIG. 7 shows the result of the analysis in GO and KEGG databases of genes whose expression decreased with drug combination in HepG2 cells.

FIG. 8 shows the result of the analysis in GO and KEGG databases of genes whose expression increased with drug combination in Hep3B cells.

FIG. 9 shows the result of the analysis in GO and KEGG databases of genes whose expression decreased with drug combination in Hep3B cells.

### DETAILED DESCRIPTION OF THE INVENTION

The objective of the present invention relates to the use of sodium pentaborate pentahydrate, curcumin, and piperine in combination as active substance for the treatment of liver cancer.

In the present invention, the term “use in combination” means the simultaneous or sequential use either together in the same formulation or in separate formulations.

In the preferred embodiment of the invention, the liver cancer is non-viral liver cancer or viral liver cancer. Preferably, this is hepatocellular carcinoma, which is a type of cancer.

Another object of the invention relates to a formulation comprising sodium pentaborate pentahydrate, curcumin, and piperine used as a drug for the treatment of hepatocellular carcinoma. This ternary combination has a higher efficacy in the treatment of hepatocellular carcinoma compared to the administration of sodium pentaborate, curcumin and piperine in the same amount and alone, or the use of a binary combination of any two of them together.

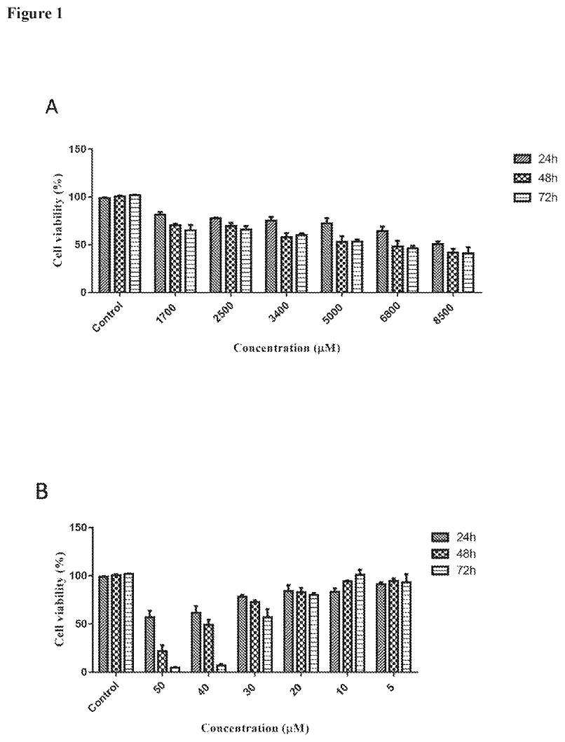

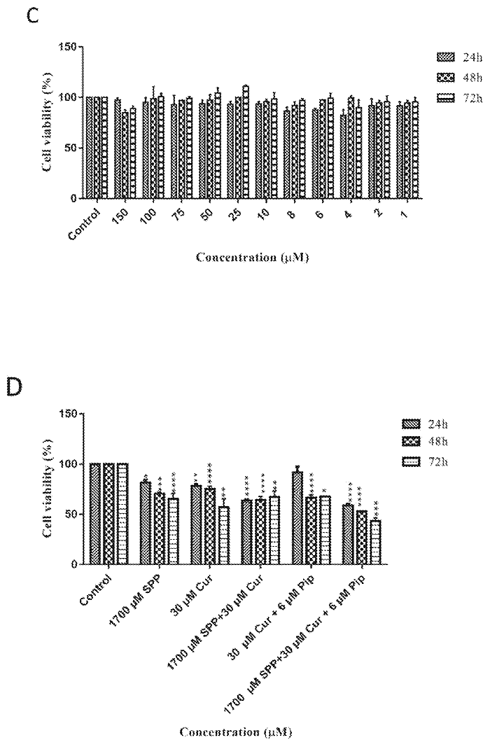

The invention has shown synergistic effect on hepatocellular carcinoma cell lines (Hep3B and HepG2) when administered as a combination of sodium pentaborate pentahydrate, curcumin, and piperine (FIG. 1 and FIG. 2). The fact that the combination index is less than 1 (CI<1) supports this synergistic effect.

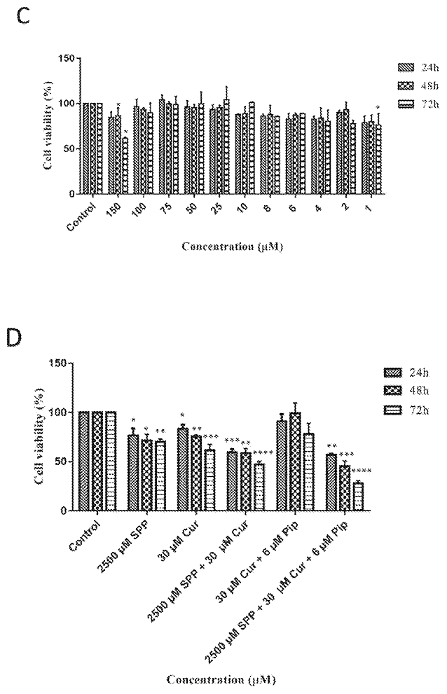

The highest percentages of cell death leading to apoptosis in HepG2 and Hep3B cells treated with sodium pentaborate pentahydrate, curcumin, and piperine were observed in the ternary combination drug administration (FIG. 4).

The said invention relates to a formulation comprising sodium pentaborate pentahydrate, curcumin, and piperine or a pharmaceutically acceptable salt or derivatives (solvate, hydrate, polymorphous or amorphous, etc.) of at least one of them.

The present invention relates to a formulation comprising a therapeutically effective amount of sodium pentaborate pentahydrate, curcumin, and piperine or a pharmaceutically acceptable salt or derivatives thereof and at least one pharmaceutically acceptable excipient.

As used herein, the phrase “therapeutically effective amount” means dose of the drug that achieves provides the specific pharmacological response for which the drug is administered to a patient in need of such treatment.

According to the preferred embodiment, the amount of sodium pentaborate pentahydrate in the total formulation is between 1700 and 8500 μM (micro molar), more preferably between 1700 and 2500 μM in concentration. Preferably, this concentration is 1700 μM in the total formulation.

According to the preferred embodiment, the amount of curcumin in the total formulation is between 5 and 500 μM (micro molar) in concentration. Preferably, this concentration is 30 μM in the total formulation.

According to the preferred embodiment, the amount of piperine in the total formulation is between 1 and 150 μM (micro molar) in concentration. Preferably, this concentration is 6 μM in the total formulation.

In the preferred embodiment of the invention, 1700 μM sodium pentaborate pentahydrate, 30 μM curcumin, and 6 μM piperine were used.

The route of administration of the formulation of the present invention is systemic or local. Systemic routes of administration are parenteral, inhalation, enteral or transdermal. Preferably, it is oral administration, and the formulation is an oral formulation.

The dosage form of the formulation of the present invention is in solid (tablet, capsule, dry powder for suspension, sachet, etc.), semi-solid or liquid (suspension, solution, emulsion, etc.) form.

Since the efficacy (concentrations per dose) provided by the ternary combination of the present invention is lower than the optimum efficacy of each drug alone, the cytotoxic effects caused by each drug are eliminated.

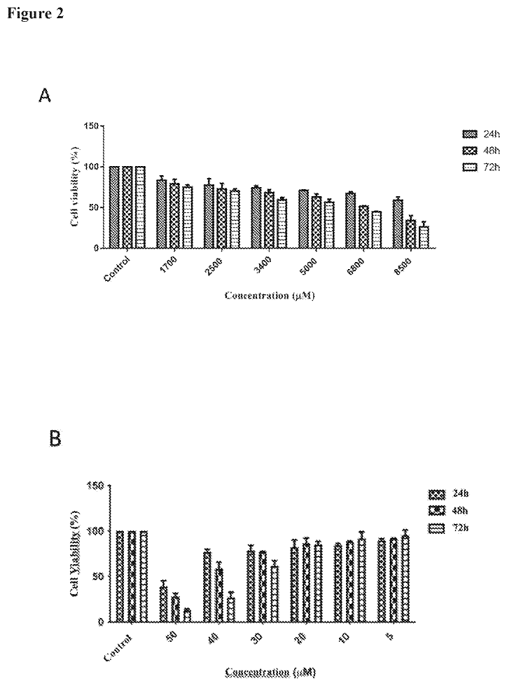

In non-cancer (HUVEC) cells, the cell inhibition rate of sodium pentaborate pentahydrate, curcumin, piperine combination was lower than that of cancer cells for 48 hours (FIG. 3).

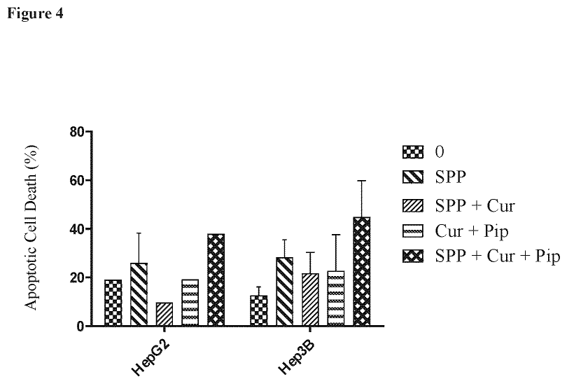

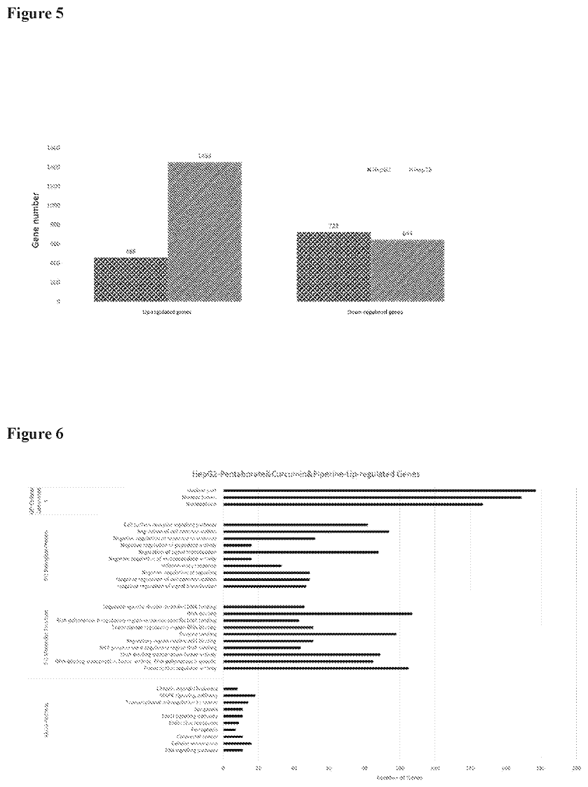

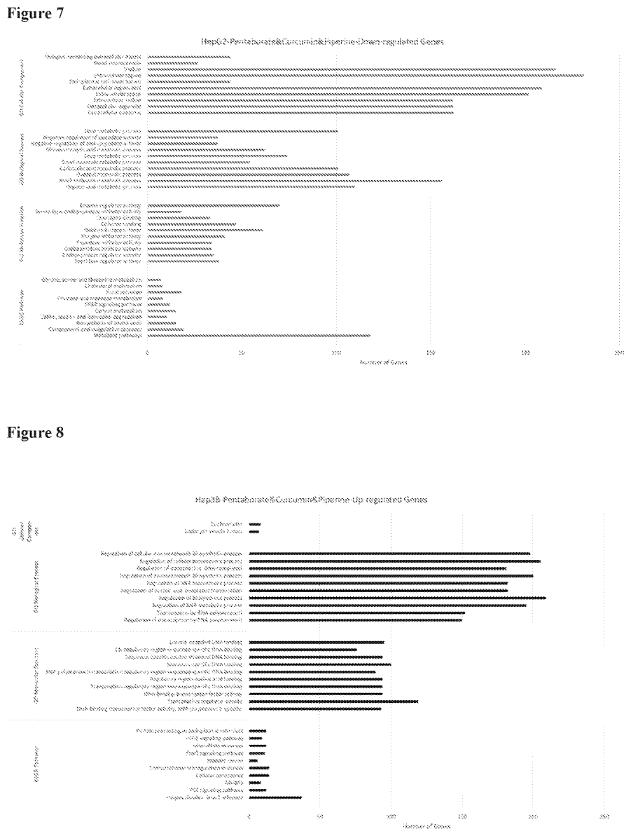

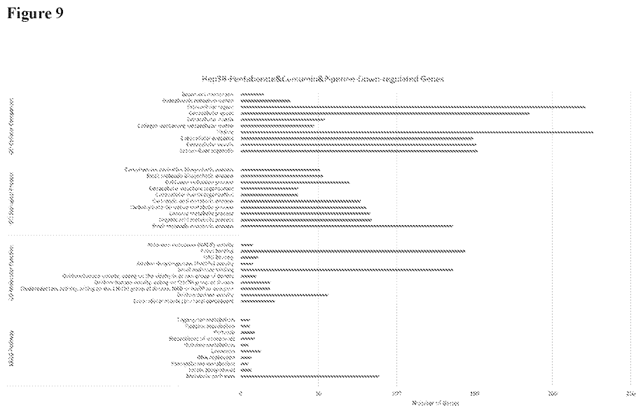

When the dysregulated genes are functionally interpreted, it is seen that they are genes that are critical for molecular function, biological process, and cellular content functions in the cell. Furthermore, pathway analysis of these genes showed that the gene expression levels of genes that are effective in cancer-related pathways such as p53, apoptosis, ferroptosis, MAPK, and FoxO signaling pathways changed (FIG. 6, FIG. 7, FIG. 8, FIG. 9).

The pharmaceutical formulations of the present invention can be prepared using standard techniques or production methods known in the state of the art.

The following examples are to better illustrate the subject of the invention and the subject of the invention is not limited to these examples.

### EXAMPLES

### Example 1—Cell Culture

Human hepatocellular cancer cell lines, HepG2 (HB-8065, ATCC), Hep3B (HB-8064, ATCC) and HUVEC (CRL-1730) cells are cultured in a growth medium (2 mM L-glutamine, 100 U/ml penicillin, 100 g/ml streptomycin) of Dulbecco's modified Eagle's medium (DMEM) containing 10% heat inactivated FBS in a humidified atmosphere at 37° C. with 5% CO₂. Cells are passaged at 3-day intervals.

### Example 2—Cell Viability Assay (Treatment of Hepatocellular Cancer Cell Lines with Sodium Pentaborate Pentahydrate, Curcumin, and Piperine)

HepG2 and Hep3B cells were seeded at 5,000 cells/well in 96-well culture dishes (Corning Glasswork, Corning, NY) in DMEM medium containing 10% FBS and 1% PSA in culture medium, then treated with Sodium pentaborate pentahydrate (NaB: B₅H₁₀NaO₁₃), Curcumin (Sigma, C1386), and Piperine (Sigma, P49007). At the end of 24, 48 and 72 hours, cell viability were measured. Cell viability assay was performed by 3-(4,5-di-methyl-thiazol-2-yl)-5-(3-carboxy-methoxy-phenyl)-2-(4-sulfo-phenyl)-2H-tetrazolium (MTS) method (CellTiter96 AqueousOne Solution; Promega, Southampton, UK) following the manufacturer's protocol. After 2 hours of incubation time, the cell viability assay is conducted by measuring at 490 nm using an ELISA microplate reader (Bio-tek ELx800, USA). IC₅₀ value for 48 hours was calculated. Accordingly, the IC₅₀ values for HepG2 were 7664.5 μM for sodium pentaborate pentahydrate and 44.8 μM for curcumin. Accordingly, the IC 50 values for Hep3B cell line were 6561 μM for sodium pentaborate pentahydrate and 41.4 μM for curcumin.

Although the dose of piperine was increased up to 150 μM, the IC₅₀ value was not reached. Cells have no toxic effects on their own. In the formulation, piperine is used as a secondary agent to enhance the bioabsorption of curcumin.

### Example 3—Apoptotic Cell Death

HepG2 and Hep3B cells were cultured at 200.000 cells/well in 6-well plates in DMEM medium containing 10% FBS 1% PSA. HepG2 cells were treated with 2500 μM Sodium pentaborate pentahydrate, 30 μM Curcumin, and 6 μM Piperine. Hep3B cells were treated with 1700 μM Sodium pentaborate pentahydrate, 30 μM Curcumin, and 6 μM Piperine. After a period of 48 hours, floating and adherent cells were collected. Cells are then incubated with Annexin V and PI stains for 15 minutes following the manufacturer's protocol of the determined Annexin V/FLOUS (Roche) apoptotic cell death kit. All samples were analyzed with 10.000 cell counts by using FACSCalibur flow cytometry (Becton Dickinson, San Jose, CA).

### Example 4—Combination Index

The combination index (CI) is used to determine the degree of drug interaction. The concentration of inhibition in the combination is the sum of the ratio of the dose of each drug to the dose of each drug used alone [28]. If the drug combination shows a synergistic effect, the CI analysis result should be CI<1. The formalization is as follows:

$$CI = [ {( D )}_{1} / {( Dx )}_{1} ] + [ {( D )}_{2} / {( Dx )}_{2} ]$$

(Dx)₁, (Dx)₂: Individual IC₅₀ values of the substances
(D)₁, (D)₂: Concentrations of substances in the combination

Accordingly, it is CI<1 for the combinations of sodium pentaborate pentahydrate and curcumin used as anti-cancer agents in HepG2 and Hep3B cell lines. The problem related to the fact that curcumin showed poor bioabsorption, which was overcome by using piperine as a secondary agent.

### Example 5—RNA Sequencing

According to the experimental results, total RNA isolation was performed with the Qiagen RNeasy Mini kit from HepG2 cells treated or untreated with the combination of 2500 μM sodium pentaborate pentahydrate, 30 μM curcumin and 6 μM piperine and from Hep3B cells treated or untreated with the combination of 1700 μM sodium pentaborate pentahydrate, 30 μM curcumin and 6 μM piperine at the optimum doses obtained for 48 hours, each with three replicates. After RNA quality was measured, the RNA was sent to Eurofins, Germany for RNA sequencing. A cDNA library for mRNA sequencing was created from each sample and sequencing was performed in 2×150 bp (base) and pair-end on Novaseq 6000 from Illumina. As a result of the sequencing, at least 60 million readings were obtained from each library.

### Example 6—Bioinformatics Analyses

The quality control of the readings obtained as a result of sequencing was performed with the FastQC program. Readings were mapped to the human reference genome (GRCh38) with the STAR program (version 2.7.0). Mapped readings were merged with the Cufflinks program (version 2.2.1) by using default parameters. Differentially expressed genes between cells treated with sodium pentaborate pentahydrate, curcumin and piperine and untreated cells used as negative control were determined by Cuffdiff (version 2.2.1). When the statistical value was based on P-value <0.05 and fold change (FC) was based on >2 and <−2 in HepG2 cells treated with sodium pentaborate pentahydrate, curcumin and piperine compared to the negative control, it was determined that a total of 1187 genes were dysregulated, 723 of which had increased expression levels and 644 had decreased expression levels. When the results of the analysis performed with Hep3B cells were evaluated; it was observed that a total of 2097 genes were dysregulated, 1453 of which had increased expression levels and 770 of which had decreased expression levels. Further bioinformatics analysis continued with these genes expressed in HepG2 and Hep3B.

### Example 7—Gene Ontology and Pathway Analyses

Genes with different expression levels in HepG2 and Hep3B cell lines (FC>2 and FC<−2, p<0.05) were mapped to molecular function, cellular content and biological function subgroups in the Gene Ontology database to identify and group the functions. In addition, pathway analysis was performed in the KEGG (Kyoto Encyclopedia of Genes and Genomes) database and in which pathways these differentially expressed genes function.

### REFERENCES

[1]. Yazici O, Bahar K. New Methods of Diagnosis and Treatment of Hepatocellular Carcinoma [Hepatosellüler Karsinomda Yeni Tani ve Tedavi Yöntemleri]Güncel Gastroenteroloji 2008; 12:47-52.
[2]. Sherman M, Burak K, Maroun J et al. Multidisciplinary Canadian consensus recommendations for the management and treatment of hepatocellular carcinoma Curr Oncol 2011; 18:228-40.
[3]. Davila J A, Morgan R O, Shaib Y, et al. Hepatitis C infection and the increasing incidence of hepatocellular carcinoma: a population-based study. Gastroenterology 2004; 127:1372-80.
[4]. Perz J F, Armstrong G L, Farrington L A, et al. The contributions of hepatitis B virus and hepatitis C virus infections to cirrhosis and primary liver cancer worldwide. J Hepatol 2006; 45: 529-38.
[5]. Singal A E, Lampertico P., Nahon P, Epidemiology and surveillance for hepatocellular carcinoma: New trends. Journal of Hepatology 2020; 72(2): 250-261.
[6]. A. Sangiovanni, G. M. Prati, P. Fasani, G. Ronchi, R. Romeo, M. Manini, et al. The natural history of compensated cirrhosis due to hepatitis C virus: A 17-year cohort study of 214 patients Hepatology 2006; 43: 1303-1310.
[7]. Ueno K, Miyazono N, Inoue H, Nishida H, Kanetsuki I, Nakajo M. Transcatheter arterial chemoembolization therapy using iodized oil for patients with unresectable hepatocellular carcinoma: evaluation of three kinds of regimens and analysis of prognostic factors. Cancer 2000; 88: 1574-81.
[8]. Das, B. C., Thapa, P., Karki, R., Schinke, C., Das, S., Kambhampati, S., & Evans, T., Boron chemicals in diagnosis and therapeutics. Future medicinal chemistry, 5(6), 653-676, 2013.
[9]. I Scorei, R., & Popa, R., Boron-containing compounds as preventive and chemotherapeutic agents for cancer. Anti-Cancer Agents in Medicinal Chemistry (Formerly Current Medicinal Chemistry-Anti-Cancer Agents), 10(4), 346-351, 2010.
[10]. Zhang T., Li G., Li S. ve ark., Asialoglycoprotein receptor targeted micelles containing carborane clusters for effective boron neutron capture therapy of hepatocellular carcinoma, Colloids and Surfaces B: Biointerfaces, Vol 182, 110397, 2019.
[11]. Lin S., Lin C., Liao J. ve ark., Therapeutic Efficacy for Hepatocellular Carcinoma by Boric Acid-mediated Boron Neutron Capture Therapy in a Rat Model, Anticancer Research, 33 (11) 4799-4809, 2013.
[12]. H. Çöpoğlu, “Investigation of the in vitro cytotoxic effects of boron on human hepatocellular carcinoma cell line, HepG2,” M. S.—Master of Science, Middle East Technical University, 2017.
[13]. Ling, X., Calinski, D., Chanan-Khan, A., Zhou, M., Li, F., Cancer cell sensitivity to bortezomib is associated with survivin expression and p53 status but not cancer cell types, Journal of Experimental and Clinical Cancer Research, 29:8, 2010.
[14]. Fujita, T., Doihara, H., Washio, K., Ino, H., Murakami, M., Naito, M., Shimizu, N., Antitumor effects and drug interactions of the proteasome inhibitor bortezomib (PS341) in gastric cancer cells, Anticancer Drugs, 18, 677-686, 2007.
[15]. Cardoso, F., Durbecq, V., Laes, J. F., Badran, B., Lagneaux, L., Bex, F., Desmedt, C., Willard-Gallo, K., Ross, J. S., Burny, A., Bortezomib (PS-341, Velcade) increases the efficacy of trastuzumab (Herceptin) in HER-2-positive breast cancer cells in a synergistic manner, Mol. Cancer Ther., 5, 3042-3051, 2006.
[16]. Selin Engur, Miris Dikmen, Kanser Tedavisinde Proteozom İnhibitörlerinin Önemi, Erciyes Üniversitesi Fen Bilimleri Enstitüsü Dergisi, 31(4):182-187, 2015.
[17]. Ganten, T. M., Koschny, R., Haas, T. L., Sykora, J., Li-Weber, M., Herzer, K. and Walczak, H. Proteasome Inhibition Sensitizes Hepatocellular Carcinoma Cells, But Not Human Hepatocytes, to TRAIL Hepatology, 42, 88-597, 2005.
[18]. Doğan, A., Demirci, S., Çağlayan, A. B., Kiliç, E., Günal, M. Y., Uslu, Ü., & Şahin, F, Sodium pentaborate pentahydrate and pluronic containing hydrogel increases cutaneous wound healing in vitro and in vivo. Biological trace element research, 162(1), 72-79, (2014).
[19]. Cebeci, E., Yüksel, B., & Şahin, F. Anti-cancer effect of boron derivatives on small-cell lung cancer. Journal of Trace Elements in Medicine and Biology, 126923, (2022).
[20]. Üstüner, B., Çimen, H, Sodium borate treatment induces metabolic reprogramming in hepatocellular carcinoma through SIRT3 activation. Turkish Journal of Biology, 40(4), 906-914, (2016).
[21]. Teiten, M. H., Gaascht, F., Eifes, S., Dicato, M., and Diederich, M. Chemopreventive potential of curcumin in prostate cancer. Genes Nutr. 5, 61-74. (2010).
[22]. Hatcher, H., Planalp, R., Cho, J., Torti, F. M., and Torti, S. V. Curcumin: from ancient medicine to current clinical trials. Cell. Mol. Life Sci. 65, 1631-1652. (2008).
[23]. Doello, K., Ortiz, R., Alvarez, P. J., Melguizo, C., Cabeza, L., and Prados, J. Latest in vitro and in vivo assay, clinical trials and patents in cancer treatment using curcumin: a literature review. Nutr. Cancer 70, 569-578. (2018).
[24]. M. Kakarala, et al. Targeting breast stem cells with the cancer preventive compounds curcumin and piperine. Breast Cancer Res. Treat., 122 (2009), pp. 777-785.
[25]. Patial, V., Mahesh, S., Sharma, S., Pratap, K., Singh, D., and Padwad, Y. S. Synergistic effect of curcumin and piperine in suppression of DENA-induced hepatocellular carcinoma in rats. Environ. Toxicol. Pharmacol. 40, 445-452. (2015).
[26]. Bolat Z B, Islek Z, Demir B N, Yilmaz E N, Sahin F, Ucisik M H. Curcumin- and Piperine-loaded emulsomes as combinational treatment approach enhance the anticancer activity of Curcumin on HCT116 colorectal cancer model. Front Bioeng Biotechnol 8:50. (2020).
[27]. Li, N.; Wen, S.; Chen, G.; Wang, S. Antiproliferative potential of piperine and curcumin in drug-resistant human leukemia cancer cells are mediated via autophagy and apoptosis induction, S-phase cell cycle arrest and inhibition of cell invasion and migration. J. BUON 25, 401-406. (2020).
[28]. Chou T C, Talalay P., Quantitative analysis of dose-effect relationships: the combined effects of multiple drugs or enzyme inhibitors. Advances in Enzyme Regulation 22, 27-55, 1984.

1. A formulation comprising the use of sodium pentaborate pentahydrate, curcumin and piperine in combination as active substance for use in treatment of liver cancer.

2. A formulation according to claim 1, wherein it is used in the treatment of non-viral liver cancer or viral liver cancer.

3. A formulation according to claim 2, wherein the liver cancer is hepatocellular carcinoma.

4. A formulation according to claim 1, wherein it comprises therapeutically effective amounts of active substances of sodium pentaborate pentahydrate, curcumin, and piperine, or a pharmaceutically acceptable salt or derivatives of at least one of them.

5. A formulation according to claim 1, wherein it comprises at least one pharmaceutically acceptable excipient.

6. A formulation according to claim 1, wherein the amount of sodium pentaborate pentahydrate in total formulation is between 1700 and 8500 μM, preferably 1700 μM in concentration.

7. A formulation according to claim 1, wherein the amount of curcumin in total formulation is between 5 and 50 μM, preferably 30 μM in concentration.

8. A formulation according to claim 1, wherein the amount of piperine in total formulation is between 1 and 150 μM, preferably 6 μM in concentration.

9. A formulation according to claim 1, wherein it comprises 1700 μM sodium pentaborate pentahydrate, 30 μM curcumin, and 6 μM piperine.

10. A formulation according to claim 1, wherein the route of administration of the formulation is systemic or local.

11. A formulation according to claim 1, wherein the route of administration of the formulation is oral administration.

12. A formulation according to claim 1, wherein the dosage form is in solid, semi-solid or liquid form.

13. A process of using a formulation according to claim 1 in a production of a drug used for a treatment of liver diseases.
